(19) United States
(12) Patent Application Publication
(10) Pub. No.: US 2026/0047535 A1
(43) Pub. Date: Feb. 19, 2026

(54) CONTROLLED ENVIRONMENT AGRICULTURE SYSTEM AND METHOD

(71) Applicant: Leftfield Urban Limited, Aberdeen (GB)

(72) Inventors: Scott SHAND, Aberdeen (GB); Euan SHAND, Aberdeen (GB)

(73) Assignee: Leftfield Urban Limited, Aberdeen (GB)

(21) Appl. No.: 18/865,822

(22) PCT Filed: May 16, 2023
(86) PCT No.: PCT/GB2023/051287

§ 371 Date: Nov. 14, 2024

(30) Foreign Application Priority Data
May 16, 2022 (GB) 2207171.6

(51) Int. Cl. A01G 31/02; A01G 18/60; A01K 61/10; A01K 67/33

(52) U.S. Cl. CPC A01G 31/021; A01G 18/60; A01G 31/065; A01K 61/10; A01K 67/33

(57) ABSTRACT

A controlled environment agriculture system and method are described, in which the system is modular and configured for retrofit location in a pre-existing building structure. The system comprises one or more multi-modal farm modules, or each multi-modal farm module comprising an aquaculture module, a plant production module, a mushroom production module and a waste regeneration module. The aquaculture module, plant production module, mushroom production module and waste regeneration module are coupled together and operatively associated to form a process loop, whereby at least one output from the aquaculture module forms an input into the plant production module, at least one output from the plant production module forms an input into the mushroom production module, at least output from the mushroom production module forms an input into the waste regeneration module, and at least one output from the waste regeneration module forms an input into the aquaculture module

### FIELD

This relates to a controlled environment agriculture system and method.

### BACKGROUND

The cultivation of plants and livestock by farming has been used for millennia in order to provide food for human and animal consumption. Over time, farming techniques and equipment have developed significantly with amongst other things advances in mechanisation and agrochemicals such as pesticides and fertilisers resulting in significant increases in yields.

Nevertheless, there remain technical and environmental challenges faced by farming and the associated supply chains.

For example, outdoor farms by their nature require the use of significant areas of land. Outdoor farms also remain dependent on the seasonality of the crops being grown and the climate in which the crops are cultivated. Despite the ability to achieve increased yields, the yield in any given growing season is vulnerable to the weather and the threat of disease and pests.

The desire to maximise efficiencies has also led to many farms adopting a monoculture whereby only one or a few products are cultivated, with consequential decreases in soil fertility and increased use of artificial pesticides and/or fertilisers.

Controlled environment agriculture systems and methods, such as growing crops in greenhouses or vertical farms, have been developed to limit crop impacts widely experienced in outdoor farming. These controlled environment agriculture systems provide benefits over outdoor farms in that they facilitate the cultivation of crops all year round and provide protection from the weather and changes in climate.

Nevertheless, conventional controlled environment agriculture systems also have drawbacks, one of the main drawbacks being that they involve significant infrastructure and costs to set up and operate.

Moreover, the supply chains for both conventional outdoor farming and controlled environment agriculture systems often involve the transportation of food and/or animal products over significant distances with the result that the production and transportation of such products to market involves a high emissions footprint. In the case of food and animal products with a short shelf life, transportation is often by air with even greater environmental impact.

### SUMMARY

Aspects of the present disclosure relate to a controlled environment agriculture system and to a method of farming using said controlled environment agriculture system.

According to a first aspect, there is provided a controlled environment agriculture system,
wherein the system is modular and configured for retrofit location in a pre-existing building structure.

In use, the system is configured for location in a pre-existing building structure, in particular but not exclusively an office block, office/warehouse combination, retail unit or the like, and is operable to provide a range of food products including plants, fish and mushrooms.

The system provides a number of significant benefits over both conventional outdoor farms and conventional controlled environment agriculture systems.

For example, the system provides a sustainable farming system that facilitates the cultivation of crops all year round and provides protection from the weather and changes in climate, without the land use requirements of conventional farming systems. The system provides for reduced reliance on food imports and the associated emissions footprint and/or provides greater food security.

The controlled indoor setting of the pre-existing building structure means that the use of pesticides can be eliminated. Moreover, there is a reduced requirement for labour, machinery and equipment in comparison with rural farms, as well as the associated costs and environmental impact.

In particular embodiments, the system's ability to be adapted to pre-existing building structures, such as office blocks, office/warehouse combinations, retail units and the like, means that the system may be located in or in close proximity to urban centres or other locations which are unsuitable for conventional controlled environment agriculture systems such as greenhouses. This in turn has the benefit that lead times to market can be reduced, resulting in longer shelf life, reduced food waste and/or a product range which is more adaptable and/or responsive to end user demand.

Moreover, the system's ability to be adapted to pre-existing building structures, such as office blocks, office/warehouse combinations, retail units and the like, means that existing building infrastructure can be re-purposed, thereby obviating the time, labour, environmental impact and/or capital expenditure associated with the construction of a purpose-built structure to house a controlled environment agriculture system.

As described above, the system is modular and configured for retrofit location in a pre-existing building structure.

The system may comprise one or more multi-modal farm modules, comprising:
an aquaculture module;
a plant production module;
a mushroom production module; and
a waste regeneration module,
wherein the aquaculture module, the plant production module, the mushroom production module and the waste regeneration module are coupled together or operatively associated to form a process loop, whereby at least one output from the aquaculture module forms an input into the plant production module, at least one output from the plant production module forms an input into the mushroom production module, at least one output from the mushroom production module forms an input into the waste regeneration module, and at least output from the waste regeneration module forms an input into the aquaculture module.

The system may be defined as an omni-ponic system.

Beneficially, the provision of a controlled environment agriculture system comprising one or more modules permits the system to be adapted for use in the pre-existing building structures. More particularly, the provision of a system comprising one or more multi-modal farm modules comprising connected aquaculture, plant production, mushroom production and waste regeneration modules, whereby at least one output from the aquaculture module forms an input into the plant production module, at least one output from the plant production module forms an input into the mushroom production module, at least one output from the mushroom production module forms an input into the waste regeneration module, and at least output from the waste regeneration module forms an input into the aquaculture module provides a self-sustaining or substantially self-sustaining system that is scalable to the footprint available in a given pre-existing building structure.

The system may be coupled to and configured to receive a feed water input. The feed water input may comprise or take the form of rainwater. The system may comprise or may be coupled to a rainwater collection system. The system may comprise or may be coupled to a filtration system. The filtration system may be configured and/or operable to filter the feed water input.

The system may comprise a fluid conduit system for communicating the feed water input to parts of the system, for example to the aquaculture module, the plant production module, the mushroom production module and the waste regeneration module. At least one fluid conduit of the fluid conduit system may, for example, comprise or take the form of a rigid pipe. Alternatively or additionally, at least one fluid conduit of the fluid conduit system may comprise or take the form of a flexible hose.

In some embodiments, in particular those in which the system is located within a multi-storey building structure, the fluid conduit system may be located or configured for location alongside the building's water system, and piping, thus minimising adaptation costs and/or the disruption associated with the installation of new water supply infrastructure to the building structure.

The system may comprise or may be coupled to an energy supply.

The energy supply may be configured and/or operable to provide an electrical power input to the system.

The energy supply may comprise or take the form of a bio-gas unit.

The bio-gas unit may be configured and/or operable to receive plant and/or organic waste from the system and convert this into electricity for supply to the system.

The energy supply may comprise or take the form of a solar power arrangement. The energy supply may comprise or take the form of one or more photovoltaic panels. In particular but not exclusively, one or more of the photovoltaic panels may comprise or take the form of a perovskite-on-silicon photovoltaic panels. However, it will be understood that any suitable solar power arrangement may be utilised.

Alternatively or additionally, the energy supply may comprise or take the form of one or more solar thermal panels. The one or more solar thermal panels may be configured and/or operable to produce heat to the system.

The energy supply may comprise or take the form of one or more wind energy capture device, e.g. one or more wind turbine.

Alternatively or additionally, the energy supply may comprise a mains electricity supply or electrical generator.

Alternatively or additionally, the energy supply may comprise or may be coupled to a battery backup system e.g. an uninterruptible power supply (UPS) arrangement. The battery backup system may comprise one or more batteries.

The energy supply may be configured to utilise 100% or substantially 100% renewable energy sources during normal operation. However, the energy supply may be coupled to a mains power supply such that power can be drawn or supplied to the mains electrical grid.

Beneficially, the energy supply may provide multiple redundancy in the event of power loss or interruption in the supply of power to the system.

As described above, the system may comprise an aquaculture module.

In use, the aquaculture module may be supplied with juvenile fish, e.g. fingerlings, and a feedstock, e.g. worms from the waste regeneration module, and produces a fish output in the form of adult fish for supply to the market.

The aquaculture module may comprise or take the form of a recirculating aquaculture system.

The aquaculture module may be coupled to and configured to receive the feed water input.

The aquaculture module may be coupled to and configured to receive the electrical power input from the energy supply.

The aquaculture module may receive a cool air input.

In use, the aquaculture module produces heat and the cool air input may be configured and/or operable to cool the aquaculture module.

The cool air input may comprise or take the form of air having a temperature below 16° Celsius.

The aquaculture module may comprise one or more water tanks.

The aquaculture module may comprise a fluid conduit system.

The fluid conduit system may comprise one or more fluid conduits configured and/or operable to supply the feed water input to the one or more water tanks and/or circulate water around the aquaculture module. At least one of the fluid conduits of the fluid conduit system of the aquaculture module may, for example, comprise or take the form of a rigid pipe. Alternatively or additionally, at least one of the fluid conduits of the fluid conduit system of the aquaculture module may comprise or take the form of a flexible hose.

The aquaculture module may comprise a water circulation system.

The water circulation system may comprise a pump arrangement. The pump arrangement may comprise one or more pump configured and/or operable to circulate water to and/or from the one or more water tanks.

The aquaculture module may comprise a filtration arrangement.

The filtration arrangement may comprise one or more filters. At least one of the filters may comprise a filter media. The filter media may comprise or take the form of an organic material, e.g. wood such as waste wood, and/or straw.

In use, the filter media absorbs nitrates from the water and/or softens the water.

The one or more filters may comprise or take the form of a replaceable filter pack.

The used filer media may, for example, be directed to another production module, e.g. the mushroom production module.

Beneficially, the wet filter media is particularly useful in the system cultivation.

As described above, the aquaculture module, the plant production module, the mushroom production module and the waste regeneration module are coupled together or operatively associated to form a process loop whereby at least one output from the aquaculture module forms an input into the plant production module, at least one output from the plant production module forms an input into the mushroom production module, at least output from the mushroom production module forms an input into the waste regeneration module, and at least output from the waste regeneration module forms an input into the aquaculture module.

The aquaculture module may be configured and/or operable to provide a waste water output. The waste water output may comprise fish waste from the fish.

Beneficially, the waste water output may comprise ammonia and/or nitrates which can be utilised in the plant production module to facilitate the growth of crops.

The waste water output from the aquaculture module may form the at least one output from the aquaculture module which forms an input into the plant production module.

The aquaculture module may be configured and/or operable to provide a warm air output.

The warm air output may comprise or take the form of air having a temperature at or above 16® Celsius. The warm air output may comprise or take the form of air having a temperature in the range 16° Celsius to 18° Celsius.

The warm air output from the aquaculture module may be directed to the plant production module. The warm air output from the aquaculture module may be directed to the mushroom production module and/or the waste regeneration module.

The warm air output from the aquaculture module may form the at least one output from the aquaculture module which forms an input into the plant production module.

The system may comprise an air handling system.

The air handling system may be configured and/or operable to communicate air, and associated component gases such as oxygen and carbon dioxide, around the system, e.g. between the aquaculture module, the plant production module, and the mushroom production module.

The air handling system may be configured and/or operable to draw in air from an exterior of the system.

The air handling system may be configured to direct the cool air output to the aquaculture module.

The air handling system may be configured to direct the warm air input from the aquaculture module.

Beneficially, the air handling system may control air flow around the system and/or facilitate temperature control by communicating warm air from areas of the system which generate excess heat to areas of the system in which said heat can be utilised, e.g. in the production of the plants in the plant production module, to facilitate the growth of mushrooms in the mushroom production module and/or to facilitate the breakdown of organic material in the waste regeneration module.

Moreover, the air handling system may be configured and/or operable to vary the temperature and/or amounts of gases such as air, and associated components gases such as oxygen and carbon dioxide, in the system or parts thereof.

Beneficially, the air handling system may be adaptable to control the temperature and/or humidity of the component parts of the system and/or amounts of gasses present in order to best suit the required conditions for that part of the system. The air handling system may for example be configured and/or operable to control the temperature and/or humidity in response to operation of the system and/or in response to external factors such as external temperature, temperature fluctuations, solar energy impinging on and/or stored within the building structure.

The air handling system may comprise a flow conduit system. The flow conduit system may be configured and/or operable to communicating air between the modules.

The air handling system may comprise or form part of an air handling system of the building structure. For example, the air handling system of the building structure may comprise or take the form of the heating, ventilation and/or air conditioning (HVAC) or equivalent system of the building structure.

Beneficially, the system is capable of utilising the air handling system from the pre-existing building structure and thus obviates the requirement for, and associated costs and/or disruption associated with, the installation of new infrastructure.

As described above, the system may comprise a plant production module.

In use, the plant production module may be supplied with seeds, bulbs and/or or juvenile plants, e.g. seedlings, and produces a plant output for supply to the market.

The plant output may comprise one or more of: fruit; vegetables; and herbs.

Beneficially, the system is configured and/or operable to produce a wide range of plants; in contrast to conventional systems which, for the reasons explained above, tend towards a monoculture plant output.

The plant production module may be configured and/or operable to receive the at least one output from the aquaculture module and provide at least one output which forms an input to the mushroom production module.

The plant production module may receive the waste water, gas such as carbon dioxide and/or warm air outputs from the aquaculture module, e.g. via the mineralisation module, the plant production module utilising these inputs to facilitate the growth of crops.

The plant production module may be coupled to and configured to receive the feed water input.

The plant production module may be coupled to and configured to receive the electrical power input from the energy supply.

The plant production module may be coupled to and configured to receive a compost input. The compost input may be received from the waste regeneration module.

As described above, the plant production module may be configured and/or operable to provide at least one output which forms an input to the mushroom production module.

The at least one output may comprise or take the form of oxygen generated by the plants grown in the plant production module.

The air handling system may be configured and/or operable to communicate the oxygen output from the plant production module to the mushroom production module.

Alternatively or additionally, the at least one output may comprise or take the form of an organic waste output from the plant production module. The organic waste output may for example comprise or take the form of one or more of: stalks; leaves; and roots from the plants being grown.

The plant production module may comprise one or more receptacles for holding the plants to be grown and a growing media, e.g. plugs and/or compost. The one or more receptacles may be ground-mounted, wall-mounted or ceiling mounted. For example, the plant production module may comprise one or more receptacle in the form of a table, growing rack or the like. Alternatively or additionally, the plant production module may comprise one or more receptacle in the form of a basket.

The receptacles may be arranged in a single tier or a plurality of tiers.

Alternatively or additionally, the plant production module may comprise one or more wires or lines from which the plants to be grown may be suspended.

The plant production may comprise a light arrangement.

The light arrangement may comprise one or more light sources. In particular embodiments, at least one of the light sources may comprise or take the form of an LED light source.

The light arrangement may be coupled to and/or powered by the energy supply.

Beneficially, this obviates or reduces the energy costs associated with providing light to facilitate the growth of the plants in the plant production module.

Alternatively or additionally, the light arrangement may comprise or form part of the lighting system of the building structure.

The plant production may comprise a design that will optimise natural light penetration.

The plant production module may be arranged so as to be expose the plants to be grown to natural daylight. For example, the plant production module may be arranged so that the plants to be grown are exposed to natural daylight via one or more windows and/or light wells in the building structure.

As described above, the system may comprise a mushroom production module.

In use, the mushroom production module may be supplied with a spawn input and a substrate input and produces a mushroom output for supply to the market.

In particular embodiments, the substrate input may comprise wood, straw, cardboard and/or paper, in particular wood, straw, cardboard and/or paper waste. The substrate input may comprise one or more recycled materials.

As described above, the substrate input may comprise filter media from the aquaculture module.

Beneficially, the wet, nitrogen rich filter media is particularly useful in the cultivation of mushrooms.

The mushroom production module may be coupled to and configured to receive the feed water input.

The mushroom production module may be coupled to and configured to receive the electrical power input from the energy supply.

The mushroom production module may receive an oxygen input. In particular, the mushroom production module may be coupled to and configured to receive the oxygen output from the plant production module.

The mushroom production module may output a spent substrate output. The spent substrate output may be directed to the waste regeneration module.

The spent substrate output may form the at least one output from the mushroom production module which forms an input into the waste regeneration module.

The mushroom production module may output a gas output. The gas output may comprise or take the form of carbon dioxide. The mushroom production module may be coupled to the plant production module. The gas output from the mushroom production module may form a gas input to the plant production module.

The system may comprise a gas handling system for communicating the gas output from the mushroom production module to the plant production module. The gas handling system may comprise a separate system to the air handling system. Alternatively, the gas handling system may form part of the air handling system.

The gas handling system may comprise a flow conduit system.

The flow conduit system may be configured and/or operable to communicate the gas, e.g. carbon dioxide and/or oxygen.

The system may further comprise a mineralisation module.

The mineralisation module may be configured and/or operable to receive the waste water output from the aquaculture module and output one or more of: carbon dioxide gas for release into the plant production module; radiant heat; a liquid fertiliser (e.g. water with fertiliser) for the plant production module; and a solid waste output, e.g. for the waste regeneration module.

The mineralisation module may output methane gas and the system may be configured, e.g. using the gas handling system, to capture the methane gas for use in other applications.

The mineralisation module may be interposed between the aquaculture module and the plant production module.

The mineralisation module may be coupled to and configured to receive the feed water input.

The mineralisation module may be coupled to and configured to receive the electrical power input from the energy supply.

The mineralisation module may utilise aerobic and anaerobic processes to filter the exchange between the aquaculture module and the plant production module and/or with the other parts of the system.

The mineralisation module permits the aquaculture module and the plant production module to be selectively decoupled; in contrast to conventional aquaponics systems which direct waste water directly through to media beds or plant beds.

Beneficially, by decoupling the aquaculture module and the plant production module, the system facilitates a better degree of control and consistent flow of the required elements through to the plant production module. The mineralisation module may also act as a buffer in the event of loss of output from the aquaculture module, e.g. for maintenance.

As described above, the system may comprise a waste regeneration module.

The waste generation module may comprise or take the form of a composting arrangement.

In use, the composting arrangement may be supplied with an organic material input and produce a compost output for supply to the plant production module.

The organic material input may, for example, comprise or take the form of:
plant waste, e.g. stalks, leaves and/or roots from the plant production module;
spent substrate output from the mushroom production module; and/or
the solid waste output from the mineralisation module.

The composting arrangement may comprise one or more composter units or the like. The composter units may define a first stage of the composting arrangement.

In the first stage, bacteria break down the organic material input into a first stage compost output.

The composting arrangement may comprise a vermi-compost arrangement. The vermi-compost arrangement may comprise one or more vermi-compost units, e.g. worm beds. The vermi-compost arrangement may define a second stage of the composting arrangement.

In the second stage, the vermi-compost arrangement is configured to receive the first stage compost output, with worms in the worm beds converting the first stage compost to a vermi-compost output.

The waste regeneration module may be coupled to and configured to receive the feed water input.

The waste regeneration module may comprise a fluid conduit arrangement for supplying the feed water input. The fluid conduit arrangement of the waste regeneration module may be coupled to or form part of the fluid conduit system for communicating the feed water input to parts of the system. The fluid conduit arrangement may comprise a hose arrangement. The fluid conduit arrangement may comprise a sprinkler system. The sprinkler system may comprise or form part of a sprinkler system of the building structure.

The waste regeneration module may be coupled to and configured to receive the electrical power input from the energy supply.

The waste regeneration module may be configured and/or operable to produce an annelid output, for example in the form of worms.

Beneficially, the annelid output, e.g. worms, produced from the waste regeneration module may be used as a feedstock for the fish in the aquaculture module.

The annelid output, e.g. worms, from the waste regeneration module may form the at least one output from the waste regeneration module which forms an input into the aquaculture module.

The waste regeneration module may be configured and/or operable to output a gas output. The gas output may comprise or take the form of carbon dioxide gas. The gas output may be directed, e.g. via a filter, to the plant production module.

The waste regeneration module may be configured and/or operable to output a methane gas output.

The system may be configured, e.g. using the gas handling system, to capture the methane gas for use in other applications.

The waste regeneration module may be configured and/or operable to output heat. The heat may be captured through heat rods.

The system may comprise one or more further module.

The one or more further module may comprise or take the form of a further plant production module, for example a herb production module.

As described above, the system is configured for retrofit location in a pre-existing building structure.

The pre-existing building structure may comprise or take the form of an office building structure. The pre-existing building structure may comprise or take the form of a retail unit.

The pre-existing building structure may comprise or take the form of a multi-storey building structure. For example, the pre-existing building structure may comprise or take the form of a multi-storey office block, office/warehouse combination, multi-story retail unit or other suitable multi-storey building structure.

Beneficially, the controlled environment provided by existing building structures has a number of features which lend themselves to the required adaptations, in particular but not exclusively availability of natural daylight, air handling systems, electrical power systems, floor plan and/or ceiling height. Moreover, the building fabric of e.g. re-enforced concrete buildings also provides benefits in terms of thermal properties, for example but not exclusively by providing a heat sink that can be utilised in the system.

According to a second aspect, there is provided a building structure comprising the controlled environment agriculture system of the first aspect.

According to a third aspect, there is provided a method of farming using the system of the first aspect.

According to a fourth aspect, there is provided a multi-modal module for a controlled environment agriculture system, comprising:
an aquaculture module;
a plant production module;
a mushroom production module; and
a waste regeneration module,
wherein the aquaculture module, the plant production module, the mushroom production module and the waste regeneration module are coupled together to form a process loop, whereby at least one output from the aquaculture module forms an input into the plant production module, at least one output from the plant production module forms an input into the mushroom production module, at least one output from the mushroom production module forms an input into the waste regeneration module, and at least output from the waste regeneration module forms an input into the aquaculture module.

According to a fifth aspect, there is provided a controlled environment agriculture system comprising one or more of the multi-modal modules of the fourth aspect.

According to a sixth aspect, there is provided a building structure comprising the controlled environment agriculture system of the fifth aspect.

According to a seventh aspect, there is provided a method of farming using the system of the fifth aspect.

The invention is defined by the appended claims. However, for the purposes of the present disclosure it will be understood that any of the features defined above or described below may be utilised in isolation or in combination. For example, features described above in relation to one of the above aspects or below in relation to the detailed description below may be utilised in any other aspect, or together form a new aspect.

### BRIEF DESCRIPTION OF THE DRAWINGS

These and other aspects will now be described by way of example with reference to the accompanying drawings, of which:

FIG. 1 shows a controlled environment agriculture system according to the present disclosure, located within a building;

FIG. 2 shows a high-level schematic view of the controlled environment agriculture system shown in FIG. 1;

FIG. 3 shows a schematic view of a multi-modal farm module of the controlled environment agriculture system shown in FIG. 1;

FIG. 4 shows a schematic view of an aquaculture module of the controlled environment agriculture system shown in FIG. 1;

FIG. 5 shows a schematic view of the plant production module of the controlled environment agriculture system shown in FIG. 1;

FIG. 6 shows a schematic view of the mushroom production module of the controlled environment agriculture system shown in FIG. 1;

FIG. 7 shows a schematic view of a mineralisation module of the controlled environment agriculture system shown in FIG. 1;

FIG. 8 shows a schematic view of the composting module of the controlled environment agriculture system shown in FIG. 1;

FIG. 9 shows a diagrammatic plan view of the controlled environment agriculture system shown in FIG. 1;

FIG. 10 shows an enlarged view of the aquaculture module of the controlled environment agriculture system shown in FIG. 9;

FIG. 11 shows an enlarged view of the plant production module of the controlled environment agriculture system shown in FIG. 9;

FIG. 12 shows an enlarged view of the mushroom production module of the controlled environment agriculture system shown in FIG. 9; and

FIG. 13 shows an enlarged view of the waste regeneration module of the controlled environment agriculture system shown in FIG. 9.

### DETAILED DESCRIPTION OF THE DRAWINGS

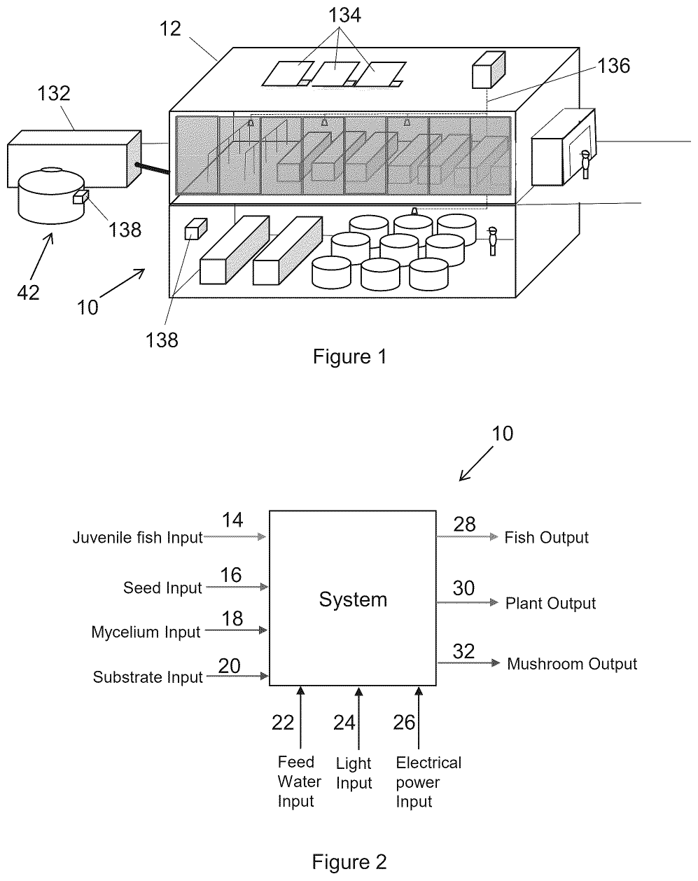

Referring first to FIGS. 1 and 2 of the accompanying drawings, there is shown a controlled environment agriculture system, generally denoted 10. As will be described further below, the system 10 is modular and configured for retrofit location in a pre-existing building structure, generally denoted 12 in FIG. 1.

The illustrated system 10 is configured for retrofit location in a pre-existing building structure 12 in the form of a multi-storey office block. However, it will be understood that the building structure 12 may take other forms such as a single storey office building structure, office/warehouse combination or a single or multi-storey retail unit.

FIG. 2 shows a high-level schematic view of the system 10. As shown, the system 10 is configured and/or operable to receive inputs in the form of a juvenile fish input 14, a seed input 16, a mycelium input 18—which in the illustrated system 10 takes the form of mushroom spawn—and a substrate input 20. The system 10 also receives a feed water input 22, a light input 24 and an electrical power input 26. The system 10 is configured and/or operable to output a range of food products including fish output 28, a plant output 30, and a mushroom output 32.

The system 10 provides a number of significant benefits over both conventional outdoor farms and conventional controlled environment agriculture systems.

For example, the system 10 provides a sustainable farming system that facilitates the cultivation of crops all year round and provides protection from the weather and changes in climate, without the land use requirements of conventional farming systems. The system 10 provides for reduced reliance on food imports and the associated emissions footprint and/or provides greater food security. The controlled indoor setting of the pre-existing building structure 12 means that the use of pesticides can be eliminated and there is a reduced requirement for labour, machinery and equipment in comparison with outdoor farms, as well as the associated costs and environmental impact.

The system's 10 ability to be adapted to pre-existing building structures 12, such as office blocks, retail units and the like, provides a number of significant advantages. For example, the system 10 can be located in or in close proximity to urban centres or other locations that are unsuitable for conventional controlled environment agriculture systems such as greenhouses and polytunnels. This in turn has the benefit that lead times to market can be reduced, resulting in longer shelf life, reduced food waste and/or a wider product range that is more adaptable and/or responsive to end user demand. Moreover, the system's 10 ability to be adapted to pre-existing building structures 12, such as office blocks, retail units and the like, means that existing building infrastructure can be re-purposed, thereby obviating the time, labour, environmental impact and/or capital expenditure associated with the construction of a purpose-built building structure to house a controlled environment agriculture system.

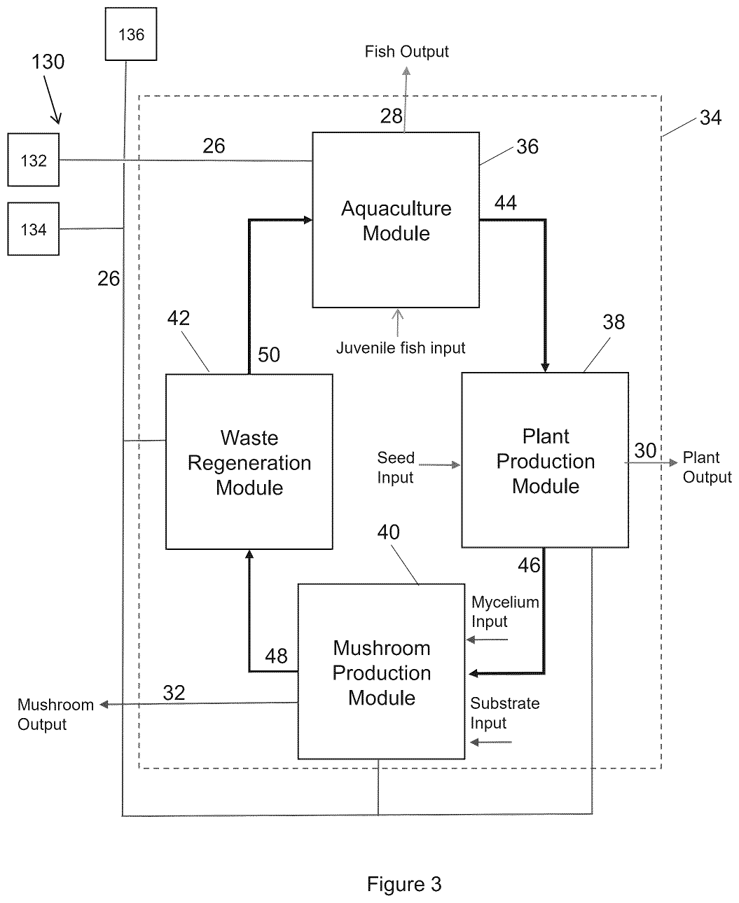

Referring now also to FIG. 3 of the accompanying drawings, the system 10 comprises one or more multi-modal farm modules, generally denoted 34, one of which is shown in FIG. 3.

As shown, the multi-modal farm module 34 comprise an aquaculture module 36, a plant production module 38, a mushroom production module 40, and a waste regeneration module 42. The aquaculture module 36, the plant production module 38, the mushroom production module 40 and the waste regeneration module 42 are coupled together or operatively associated to form a process loop, whereby at least one output 44 from the aquaculture module 36 forms an input into the plant production module 38, at least one output 46 from the plant production module 38 forms an input into the mushroom production module 40, at least one output 48 from the mushroom production module 40 forms an input into the waste regeneration module 42, and at least one output 50 from the waste regeneration module 42 forms an input into the aquaculture module 36.

In use, the multi-modal farm module 34 forms the basis of the modularity of the system 10, with the number of multi-modal farm modules 34 selected according to the space available in the building structure 12 and/or the desired yield from the system 10. Notably, each multi-modal farm module 34 forms a self-sustaining or substantially self-sustaining farm system with the inputs and outputs from each of the aquaculture module 36, the plant production module 38, the mushroom production module 40 and the waste regeneration module 42 configured to ensure that the required amounts of nutrients and/or energy are transferred to/from each part of the system 10.

Beneficially, the provision of a controlled environment agriculture system 10 comprising the one or more modules 34 permits the system 10 to be adapted for use in the pre-existing building structures 12, while providing a self-sustaining or substantially self-sustaining system 10 that is scalable to the footprint available in the given pre-existing building structure 12 and which reduces the amount of external inputs required to be supplied to the system 10 after the system 10 has been installed and commissioned; in contrast to conventional controlled environment agriculture systems which require high levels of inputs in order to operate.

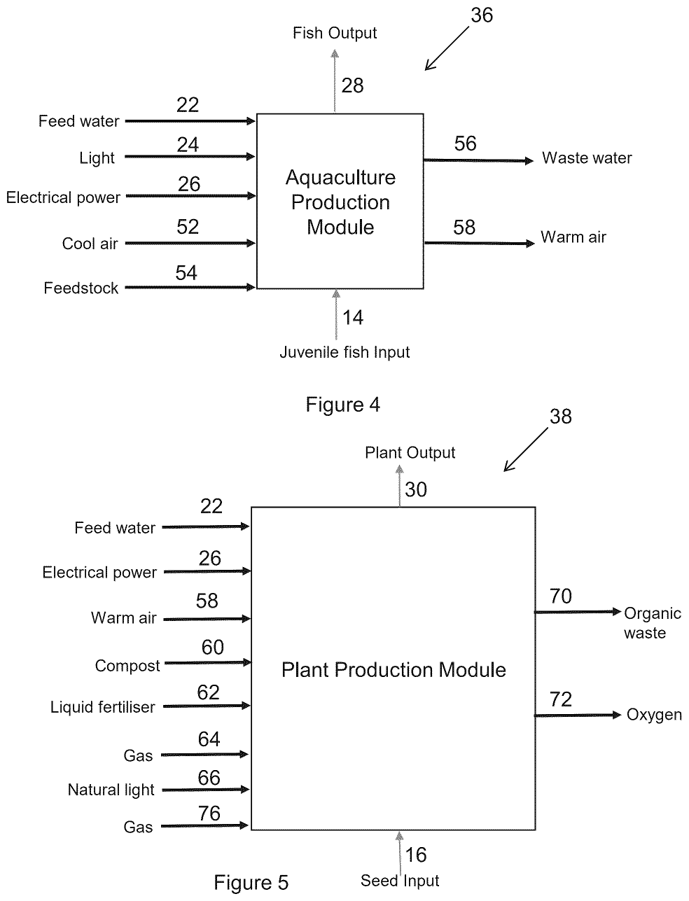

FIG. 4 of the accompanying drawings shows a schematic view of the aquaculture module 36 of the system 10.

As shown in FIG. 4, inputs into the aquaculture module 36 comprise the juvenile fish input 14, which in the illustrated system 10 comprises or takes the form of fingerlings, the feed water input 22, the light input 24, the electrical power input 26, a cool air input 52, which in the illustrated system 10 comprises or takes the form of air having a temperature below 16° Celsius, and a feedstock 54, which in the illustrated system 10 comprises or takes the form of worms produced by the system 10 as will be described below.

As shown in FIG. 4, the aquaculture module 36 is configured and/or operable to produce the fish output 28 for supply to the market. The aquaculture module 36 is also configured and/or operable to output a waste water output 56, which in the illustrated system 10 comprises fish waste from the fish in the aquaculture module 36, and a warm air output 58, which in the illustrated system 10 comprises or takes the form of air having a temperature in the range of 16° Celsius to 18° Celsius.

Beneficially, the waste water output 56, which comprises ammonia and/or nitrates, and the warm air output 58 can be utilised in the plant production module 38 to facilitate the growth of the plants/crops forming the plant output 30. The warm air output 58 can also be utilised in the mushroom production module 40 to facilitate the growth of the mushroom output 32. The warm air output 58 can also be utilised in the waste generation module 40 to facilitate the breakdown of organic material to facilitate the formation of a compost output 60.

It will be recognised that the waste water output 56 and the warm air output 58 from the aquaculture module 36 form the at least one output 44 from the aquaculture module 36 which forms the input into the plant production module 38, reducing the need to supply water and/or power from external sources.

FIG. 5 of the accompanying drawings shows a schematic view of the plant production module 38 of the system 10.

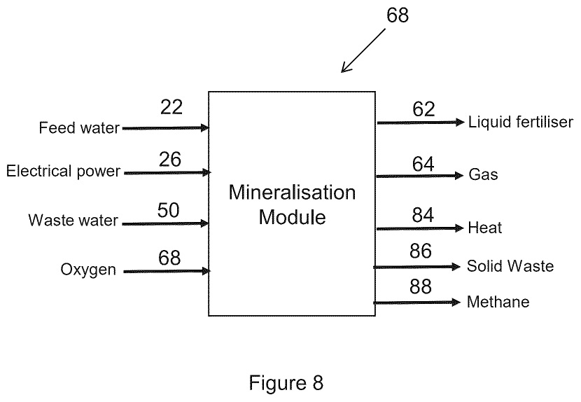

As shown in FIG. 5, inputs into the plant production module 38 comprise the seed input 16, which in the illustrated system 10 comprises seeds, bulbs and/or juvenile plants in the form of seedlings, the feed water input 22, and the electrical power input 26. The plant production module 38 also receives the warm air output 58 from the aquaculture module 36. The plant production module 38 also receives inputs in the form of liquid fertiliser (e.g. water containing fertiliser) 62, gas including carbon dioxide 64 and natural light 66. The liquid fertiliser 62 and gas 64 are directed to the plant production module 38 from the aquaculture module 36 via a mineralisation module, generally denoted 68 (shown in FIG. 8), of the system 10. The plant production module 38 also receives the compost input 60 from the waste regeneration module 42.

As shown in FIG. 5, the plant production module 38 is configured and/or operable to produce the plant output 30 for supply to the market.

Beneficially, the system 10 is configured and/or operable to produce a wide range of plants comprising one or more of: fruit; vegetables; and herbs; in contrast to conventional systems which, for the reasons explained above, tend towards a monoculture plant output.

The plant production module 38 is also configured and/or operable to output an organic waste output 70, which in the illustrated system 10 comprises stalks, leaves, and roots from the plants being grown in the plant production module 38, and an oxygen output 72 generated by the plants grown in the plant production module 38.

The oxygen output 72 from the plant production module 38 is directed to the mushroom production module 40, and it will be recognised that the oxygen output 72 forms the at least one output 46 from the plant production module 38 which forms the input into the mushroom production module 40.

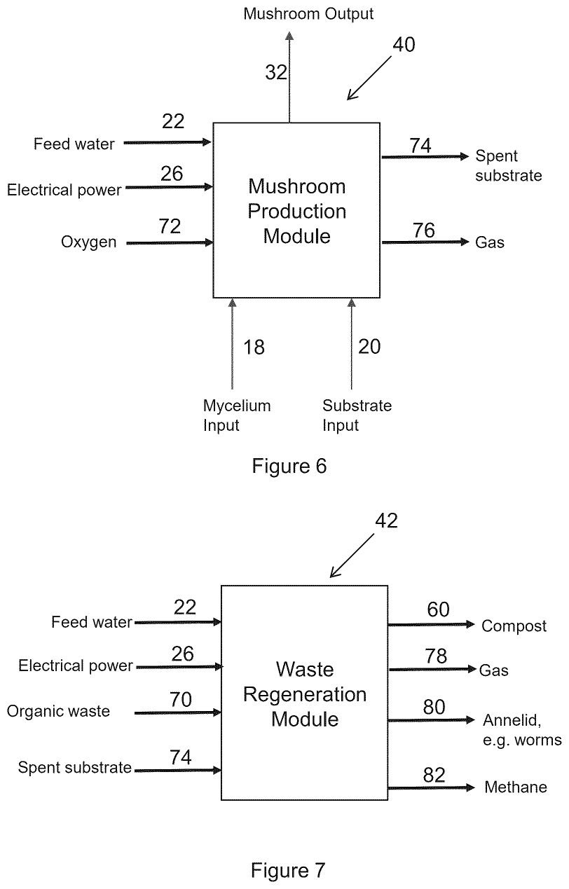

FIG. 6 of the accompanying drawings shows a schematic view of the mushroom production module 40 of the system 10.

As shown in FIG. 6, inputs into the mushroom production module 40 comprise the mycelium input 18, which in the illustrated system 10 comprises or takes the form of mushroom spawn, and the substrate input 20. In the illustrated system 10, the substrate input 20 comprises wood, straw, cardboard and/or paper filter media, and in particular wood, straw, cardboard and/or paper waste, taken from the aquaculture module 36. In the illustrated system 10, the substrate input 20 also comprises compost 60 from the waste regeneration module 42.

Beneficially, the wet, nitrogen rich filter media is particularly useful in the cultivation of mushrooms.

The mushroom production module 40 also receives the feed water input 22 and the electrical power input 26.

The mushroom production module 40 also receives the oxygen output 72 from the plant production module 38, the oxygen output 72 from the plant production module 38 forming the at least one output 46 from the plant production module 38 which forms an input into the mushroom production module 40.

As shown in FIG. 6, the mushroom production module 40 is configured and/or operable to produce the mushroom output 32 for supply to the market.

The mushroom production module 40 also outputs a spent substrate output 74 and a gas output 76. The gas output 76 comprises or takes the form of carbon dioxide. The spent substrate output 74 is directed to the waste regeneration module 42, the spent substrate output 74 from the mushroom production module 40 forming the at least one output 48 from the mushroom production module 40 which forms an input into the waste regeneration module 42.

The gas output 76 from the mushroom production module 40 is directed to and forms an input into the plant production module 38.

As described above, and referring now also to FIG. 7 of the accompanying drawings, the system 10 further comprises waste regeneration module 42.

As shown in FIG. 7, inputs to the waste regeneration module 42 comprise the feed water input 22, the electrical power input 26, the spent substrate output 74 and the organic waste output 70 from the plant production module 38.

In use, the waste regeneration module 42 is configured and/or operable to produce the compost output 60 for supply to the plant production module 38.

The waste regeneration module 42 is also configured and/or operable to produce a gas output 78 for supply to the plant production module 38, and an annelid output 80 in the form of worms for supply to the aquaculture module 36.

The waste regeneration module 42 may output methane gas 82 and the system 10 may be configured to capture the methane gas 82 for use in other applications.

As described above, and referring now also to FIG. 8 of the accompanying drawings, the system 10 comprises mineralisation module 68.

The mineralisation module 68 is interposed between the aquaculture module 36 and the plant production module 38.

The mineralisation module 68 utilises aerobic and anaerobic processes to filter the exchange between the aquaculture module 36 and the plant production module 38 and/or with other parts of the system 10.

The mineralisation module 68 permits the aquaculture module 36 and the plant production module 38 to be selectively decoupled; in contrast to conventional aquaponics systems which direct waste water directly through to media beds or plant beds.

Beneficially, by decoupling the aquaculture module 36 and the plant production module 38, the system 10 facilitates a better degree of control and consistent flow of the required elements through to the plant production module 38. The mineralisation module 68 may also act as a buffer in the event of loss of output from the aquaculture module 36, e.g. for maintenance.

As shown in FIG. 8, inputs to the mineralisation module 68 comprise the feed water input 22, the electrical power input 26, the waste water output 56 from the aquaculture module 36, and the oxygen output 72 from the plant production module 38.

As described above, the mineralisation module 68 is configured and/or operable to output the liquid fertiliser (e.g. water containing fertiliser) 62, and gas output 62.

The mineralisation module 68 is also configured and/or operable to provide outputs in the form of radiant heat 84 and a solid waste output 86 that is directed to and forms and input into the waste regeneration module 42.

The mineralisation module 68 may output methane gas 88 and the system 10 may be configured to capture the methane gas 88 for use in other applications.

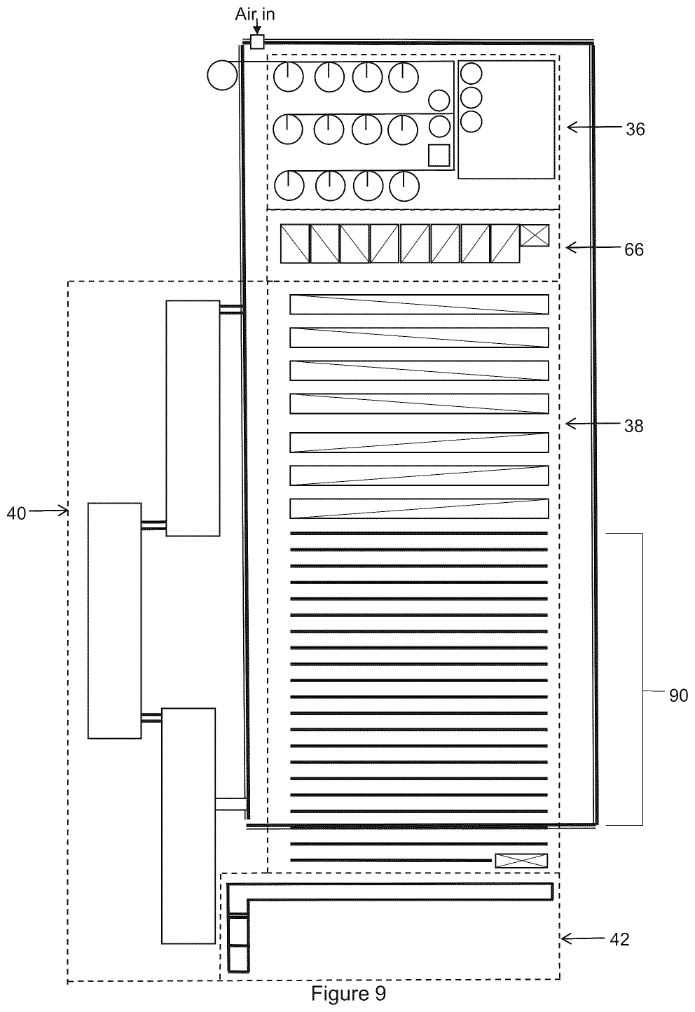

Referring now to FIG. 9 of the accompanying drawings, there is shown a diagrammatic plan view of the controlled environment agriculture system 10 shown in FIGS. 1 to 8.

As shown in FIG. 9, the system 10 is configured for retrofit location in pre-existing building structure 12 in the form of a multi-storey office block. However, it will be understood that the building structure 12 may take other forms such as a single storey office building structure, or a single or multi-storey retail unit.

As shown in FIG. 9, the system 10 comprises aquaculture module 36, plant production module 38, mushroom production module 40, and waste regeneration module 42, the aquaculture module 36, plant production module 38, mushroom production module 40 and waste regeneration module 42 forming the multi-modal farm module 34 of the system 10.

As described above, the multi-modal farm module 34 forms the basis of the modularity of the system 10, with the number of multi-modal farm modules 34 selected according to the space available in the building structure 12 and/or the desired yield from the system 10. Notably, each multi-modal farm module 34 forms a self-sustaining or substantially self-sustaining farm system with the inputs and outputs from each of the aquaculture module 36, the plant production module 38, the mushroom production module 40 and waste regeneration module 42 configured to ensure that the required amounts of nutrients are transferred to/from each part of the system 10.

The system 10 is based on a floor plan measuring 47 metres long by 20 metres wide, thereby providing a total area of 940 m² (˜10,000 ft²). In the illustrated system 10, the aquaculture module 36 occupies an area of 180 m², and the plant production module measures occupies an area of 680 m², of which 300 m² is utilised to cultivate crops in the form of leafy green vegetables and 380 m² is utilised to cultivate crops grown using vine lines 90.

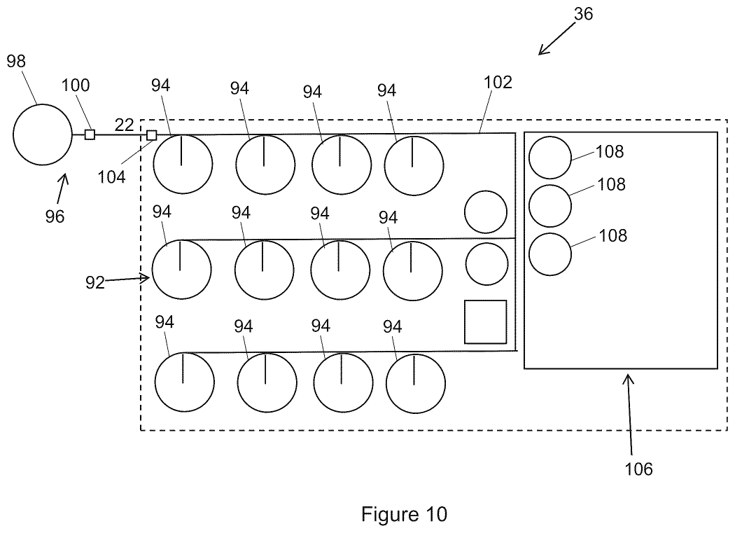

FIG. 10 of the accompanying drawings shows an enlarged view of the aquaculture module 36 of the system 10, which in the illustrated system 10 comprises or takes the form of a recirculating aquaculture system.

As shown in FIG. 10, the aquaculture module 36 comprises a water tank arrangement, generally denoted 92, comprising a plurality of water tanks 94.

The aquaculture module 36 is coupled to and configured to receive the feed water input 22, which in the illustrated system 10 comprises rainwater from a rainwater collection system 96. The rainwater collection system 96 comprises a water tank 98, which in the illustrated system 10 is capable of storing up to 10,000 Litre of water, and a filtration system 100 configured and/or operable to filter the rainwater before it enters the aquaculture module 36.

Beneficially, the system 10 is capable of utilising its own water supply, and thus obviates the requirement for, and associated costs and/or disruption associated with, the installation of new water supply infrastructure to the system 10.

As shown in FIG. 10, the aquaculture module 36 comprises a fluid conduit system 102 for supplying the feed water input 22 to the water tanks 94 and a pump arrangement 104 configured and/or operable to circulate water around the aquaculture module 36.

In the illustrated system 10, the fluid conduit system 102 comprises rigid pipe. However, it will be understood that at least part of the fluid conduit system 102 may alternatively or additionally comprise or take the form of a flexible hose.

As shown in FIG. 10, the aquaculture module 36 further comprises a filtration arrangement, generally denoted 106, comprising one or more filters 104 (three filters 108 are shown in FIG. 10).

In the illustrated system 10, the filters 108 comprise or take the form of replaceable filter packs and comprise a filter media. In use, the filters 108 absorb elements, e.g. nitrates from the water and/or where required soften the water, by removing high levels of elements that would otherwise harm the plants.

As described above, the used filer media may be directed to the mushroom production module 40, since the wet, nitrogen rich filter media is particularly useful in the cultivation of mushrooms.

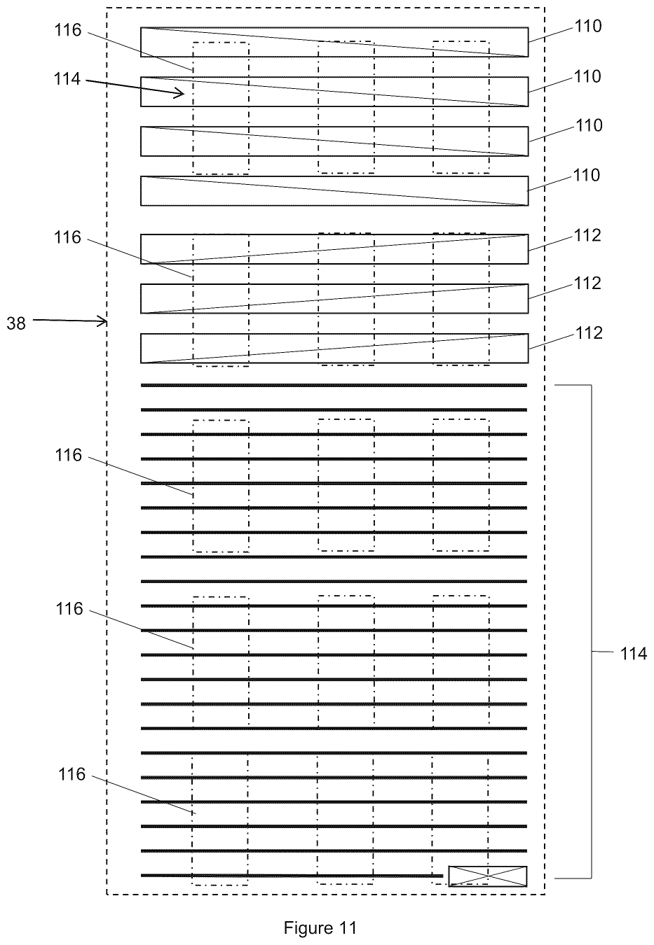

FIG. 11 of the accompanying drawings shows an enlarged view of the plant production module 38 of the system 10.

As shown in FIG. 11, the plant production module 38 comprises one or more means 90, 110, 112 for holding and/or supporting the plants to be grown.

In the illustrated system 10, the plant production module 38 comprises the vine lines 90, racks 110 for holding and/or supporting nursery plants, and growing tables 112 for cultivating leafy green vegetables.

The plant production module 38 comprises a lighting arrangement, generally denoted 114, comprising one or more light sources 116.

In the illustrated system 10, the light sources 116 comprise or take the form of an LED light sources, more particularly the LED light sources which form part of the lighting system of the building structure 12.

Beneficially, the system 10 is capable of utilising the lighting system from the pre-existing building structure 12 and thus obviates the requirement for, and associated costs and/or disruption associated with, the installation of new lighting infrastructure.

The plant production module 38 is also arranged so as to be expose the plants being cultivated to natural daylight (represented by 66 in FIG. 5). For example, the plant production module 38 is arranged so that the plants are exposed to natural daylight 66 via one or more window and/or light well in the building structure 12.

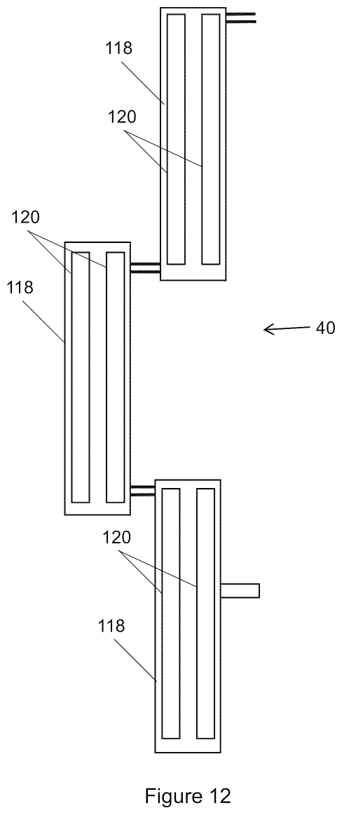

FIG. 12 shows an enlarged view of the mushroom production module 40 of the controlled environment agriculture system shown in FIG. 9.

As shown in FIG. 12, the mushroom production module 40 comprises a number of container units 118 (three container units 118 are shown).

In the illustrated system 10, each container unit 118 comprises units 120 each comprising two or three layers for growing the mushrooms. For example, in the illustrated system 10, the mushroom production module 40 provides a volume of 290 m³ for cultivating mushrooms.

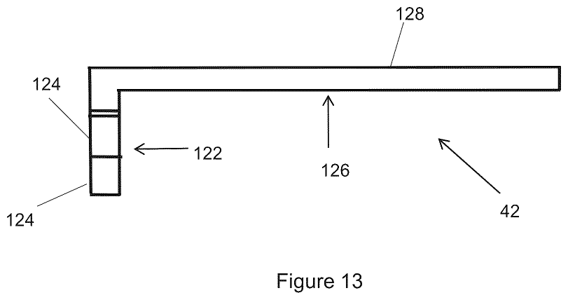

FIG. 13 of the accompanying drawings shows an enlarged view of the waste regeneration module 42 of the system 10.

As shown in FIG. 13, the waste generation module 42 comprises a first stage composting arrangement, generally denoted 122, comprising one or more composter units 124 (two composter units 124 are shown) and a second stage which in the illustrated system 10 comprises or takes the form of a vermi-compost arrangement 126.

In the first stage, bacteria breaks down the organic waste 70 and spent substrate 74 inputs into the waste regeneration module 42 before it is passed to the second stage.

The vermi-compost arrangement 126 comprises one or more vermi-compost units 128, which in the illustrated system 10 take the form of worm beds.

In use, the vermi-compost arrangement 126 receives the first stage compost output from the composter units 124 and outputs a vermi-compost output, which forms the compost output 60. Worms from the vermi-compost arrangement 126 are supplied to the aquaculture module 36, forming, or forming part of, the feedstock 54 for the fish in the aquaculture module 36.

Referring again to FIGS. 1 and 3 of the accompanying drawings, the system 10 comprises or is coupled to an energy supply, generally denoted 130, configured and/or operable to provide the electrical power input 26.

As shown, the energy supply 130 comprises a bio-gas unit 132, and one or more photovoltaic panels 134 (three panels 134 are shown by way of example in FIG. 1). The bio-gas unit 132 is configured and/or operable to receive plant and/or organic waste from the system 10 and convert this into electricity for supply to the system 10. One or more of the photovoltaic panels 134 may comprise or take the form of a perovskite-on-silicon photovoltaic panels, or other suitable PV panel.

The energy supply 124 is configured to utilise 100% or substantially 100% renewable energy sources during normal operation. However, the energy supply 124 is coupled to the mains power supply 136 such that power can be drawn from the mains electrical grid if required.

Beneficially, the energy supply 130 of the system 10 provides multiple redundancy in the event of power loss or interruption in the supply of power to the system 10.

It will be recognised that the energy supply 130 may comprise means for generating electricity from other sources. For example, the energy supply 130 may comprise or may be coupled to a wind energy capture device, generator, or other suitable power source.

As shown diagrammatically in FIG. 1, the system 10 comprises an air handling system 136.

The air handling system 136 is configured and/or operable to communicate air, and associated component gases such as oxygen and carbon dioxide, around the system 10, e.g. between the aquaculture module 36, the plant production module 38, and the mushroom production module 40. In the illustrated system 10, the air handling system 136 forms part of the air handling system of the building structure 12.

As shown diagrammatically in FIG. 1, the system 10 comprises a gas handling system 138.

The gas handling system 138 is configured and/or operable to capture the methane gas 82, 88 for use in other applications.

It will be understood that various modifications may be made without departing from the scope of the claimed invention.

For example, the system 10 may comprise one or more further module, e.g. one or more herb production modules.

1. A controlled environment agriculture system, wherein the system is modular and configured for retrofit location in a pre-existing building structure.

2. The controlled environment agriculture system of claim 1, comprising one or more multi-modal farm modules, the or each multi-modal farm module comprising:
an aquaculture module;
a plant production module;
a mushroom production module; and
a waste regeneration module,
wherein the aquaculture module, the plant production module, the mushroom production module and the waste regeneration module are coupled together or operatively associated to form a process loop whereby at least one output from the aquaculture module forms an input into the plant production module, at least one output from the plant production module forms an input into the mushroom production module, at least one output from the mushroom production module forms an input into the waste regeneration module, and at least output from the waste regeneration module forms an input into the aquaculture module.

3. The controlled environment agriculture system of claim 1, wherein the system is coupled to and configured to receive a feed water input,
and wherein optionally the feed water input comprises or takes the form of rainwater from a rainwater collection system.

4. (canceled)

5. The controlled environment agriculture system of claim 1, wherein the system is coupled to and configured to receive an electrical power input from an energy supply,
and wherein optionally the energy supply comprises or takes the form of a renewable energy power generation module comprising:
one or more photovoltaic panels;
one or more solar thermal panels;
one or more wind energy capture device, e.g. one or more wind turbine.
one or more bio-gas unit.

6. (canceled)

7. The controlled environment agriculture system of claim 2, wherein the aquaculture module comprises or takes the form of a recirculating aquaculture system.

8. The controlled environment agriculture system of claim 2, wherein the aquaculture module comprises a filtration arrangement comprising one or more filters, and wherein at least one of the filters comprises or takes the form of a replaceable filter pack,
and wherein optionally at least one of the filters comprises a filter media which comprises or takes the form of wood, e.g. waste wood, and/or straw.

9. (canceled)

10. The controlled environment agriculture system of claim 2, wherein the aquaculture module is configured and/or operable to provide at least one of:
a waste water output, e.g. comprising fish waste from fish in the aquaculture module; and
a warm air output.

11. The controlled environment agriculture system of claim 1, wherein the system comprises an air handling system,
and wherein optionally the air handling system comprises or forms part of an air handling system of the building structure.

12. (canceled)

13. The controlled environment agriculture system of claim 10, wherein the system comprises an air handling system,
and wherein the air handling system is configured and/or operable to communicate the warm air output from the aquaculture module to the plant production module, the mushroom production module and/or the waste regeneration module.

14. The controlled environment agriculture system of claim 2, further comprising a mineralisation module, wherein the mineralisation module is configured and/or operable to output one or more of:
gas for release into the plant production module;
radiant heat;
a liquid fertiliser for the plant production module; and
a solid waste output, e.g. for the waste regeneration module.

15. The controlled environment agriculture system of claim 2, wherein the plant production module is configured and/or operable to provide at least one of:
an oxygen output, the oxygen output forming the at least output from the plant production module forming the input to the mushroom production module; and
an organic waste output, the organic waste output forming the at least output from the plant production module which forms the input to the waste regeneration module.

16. The controlled environment agriculture system of claim 15, wherein the system comprises an air handling system,
and wherein the air handling system is configured and/or operable to communicate the oxygen output from the plant production module to the mushroom production module.

17. The controlled environment agriculture system of claim 15, wherein the system comprises an air handling system.
and wherein the air handling system is configured and/or operable to communicate the oxygen output from the plant production module to the mineralisation module.

18. The controlled environment agriculture system of claim 2, wherein the plant production module comprises a light arrangement,
and wherein optionally the light arrangement comprises or form part of a lighting system of the building structure.

19. (canceled)

20. The controlled environment agriculture system of claim 2, wherein the plant production module is arranged so as to be expose the plants to be grown to natural daylight.

21. The controlled environment agriculture system of claim 2, wherein the mushroom production module is configured to output at least one of:
a spent substrate output; and
a gas output.

22. The controlled environment agriculture system of claim 2, wherein the waste regeneration module comprises a composting arrangement, wherein the composting arrangement is configured and/or operable to output a compost output for supply to the plant production module,
and wherein optionally the composting arrangement comprises a vermi-compost arrangement, the vermi-compost arrangement configured and/or operable to produce an annelid output for supply to the aquaculture module.

23. (canceled)

24. The controlled environment agriculture system of claim 2, wherein the system comprises one or more further module coupled to the multi-modal module, wherein the at least one of the one or more further modules optionally comprises or takes the form of a herb production module.

25. A building structure comprising the controlled environment agriculture system of claim 1,
wherein optionally the building structure comprises or takes the form of a multi-storey building structure.

26. (canceled)

27. A method of farming using the controlled environment agriculture system of claim 1, comprising:
providing the controlled environment agriculture system; and
operating the controlled environment agriculture system to produce a fish output, a plant output and a mushroom output.

28. A multi-modal module for a controlled environment agriculture system, comprising:
an aquaculture module;
a plant production module;
a mushroom production module; and
a waste regeneration module,
wherein the aquaculture module, the plant production module, the mushroom production module and the waste regeneration module are coupled together to form a process loop whereby at least one output from the aquaculture module forms an input into the plant production module, at least one output from the plant production module forms an input into the mushroom production module, at least one output from the mushroom production module forms an input into the waste regeneration module, and at least output from the waste regeneration module forms an input into the aquaculture module.

29. A controlled environment agriculture system comprising one or more of the multi-modal modules of claim 28.

30. A building structure comprising the controlled environment agriculture system of claim 29,
wherein optionally the building structure comprises or takes the form of a single or multi-storey building structure.

31. (canceled)

32. A method of farming using the controlled environment agriculture system of claim 28 comprising:
providing the controlled environment agriculture system; and
operating the controlled environment agriculture system to produce a fish output, a plant output and a mushroom output.
